(19) United States
(12) Patent Application Publication
(10) Pub. No.: US 2026/0306785 A1
(43) Pub. Date: Oct. 8, 2026

(54) AI-POWERED NEUROPLASTICITY VIA WEARABLE AND EMBEDDED BRAIN INTERFACES

(71) Applicants: Massimiliano Moruzzi, Rockford, IL (US); Brenda Affinati, North Chicago, IL (US); Jeremy Rosenkranz, North Chicago, IL (US); Grace Stutzmann, North Chicago, IL (US); Marc Glucksman, North Chicago, IL (US); Holly Hunsberger, North Chicago, IL (US); Ana Sanfilippo, North Chicago, IL (US); Joshua Affinati, North Chicago, IL (US); Marco Andriano, Bloomfield Hills, MI (US)

(72) Inventors: Massimiliano Moruzzi, Rockford, IL (US); Brenda Affinati, North Chicago, IL (US); Jeremy Rosenkranz, North Chicago, IL (US); Grace Stutzmann, North Chicago, IL (US); Marc Glucksman, North Chicago, IL (US); Holly Hunsberger, North Chicago, IL (US); Ana Sanfilippo, North Chicago, IL (US); Joshua Affinati, North Chicago, IL (US); Marco Andriano, Bloomfield Hills, MI (US)

(73) Assignee: Neurobolt.ai, North Chicago, IL (US)

(21) Appl. No.: 19/641,111

(22) Filed: Apr. 7, 2026

Related U.S. Application Data
(60) Provisional application No. 63/784,758, filed on Apr. 7, 2025.

(51) Int. Cl. A61M 21/00; A61N 2/00

(52) U.S. Cl. CPC A61M 21/00; A61N 2/006; A61M 2021/0055; A61M 2021/0072; A61M 2230/10

(57) ABSTRACT

Embodiments of the disclosure relate to an AI-powered neuroplasticity device via wearable and embedded brain interfaces. AI-Powered Neuroplasticity via Wearable and Embedded Brain Interfaces harnesses the brain's natural ability to rewire itself, termed, neuroplasticity, by using adaptive AI to analyze, decode, translate, predict, and modulate neural patterns and deliver, in real-time, temporospatial, personalized, and adaptive feedback via wearable or embedded brain-computer interfaces (BCIs).

### CROSS-REFERENCE TO RELATED PATENT APPLICATIONS

This patent application claims the benefit of U.S. Provisional Patent Application No. 63/784,758, filed Apr. 7, 2025, the entire teachings and disclosure of which are incorporated herein by reference thereto.

### FIELD OF THE INVENTION

This invention generally relates to wearable and embedded brain interfaces and, in particular, to AI-powered neuroplasticity using such interfaces.

### BACKGROUND OF THE INVENTION

Neuroplasticity is a process within the brain that involves adaptive structural and functional changes in response to new experiences, injury, environments, and many other stimuli. In fact, humans make decisions, form memories, and produce behaviors through a finely tuned balance of neuronal activity. Disruption in this neuronal activity underlies many neurological and psychiatric disorders. For example, neurodegenerative diseases, such as Alzheimer's, Parkinson's, and ALS, result in the gradual loss of cognitive or motor neuron function, leading to a significant decline in quality of life. At the same time, the ability to augment, enhance, or expand normal human performance through neurofeedback is increasingly seen as an opportunity to unlock human potential. Therefore, the most promising approach to repair brain function and augment the brain to enhance performance is to leverage neuronal plasticity.

However, because the brain-wide connectome is not fully understood (i.e., how specific connections lead to specific behaviors), it is difficult to 1) treat or prevent neurological disorders, and 2) enhance brain performance. Currently, not enough is known about individual neuronal activity patterns that can promote plasticity nor can these patterns be reproduced in individual neurons without causing significant damage to the neuronal environment. While there are databases that contain connections and neuronal activity data, they are problematic because 1) they are disconnected from behavior and would not be helpful for transferring appropriate patterns to neurons, and 2) there is a lack of compatible AI data. Additionally, current brain implants are not viable and lead to scar tissue, infection, and an inflammatory response. This changes the entire microenvironment function of the tissue and chemical and electrophysiological signals can become unstable and inaccurate. This is significant because the brain needs to remain flexible to send biological feedback information to the wearable device so that an AI interface can also make adjustments.

In the context of personalized and precision medicine, there is an urgent need for approaches that are tailored to the specific physiology, biology, and long-term functionality of the individual. These solutions must also integrate and leverage the individual's unique neural data to provide optimal outcomes over time. The complexity of this challenge is underscored by the brain's staggering architecture: approximately 90 billion neurons forming 100 trillion synaptic connections, each influencing the intricate dynamics of brain function. AI interactive devices would customize interventions based on each individual's brain activity, offering highly effective and targeted neuroplastic stimulation (i.e., personalized medicine).

Applicant has recognized a need for a more durable and biocompatible electrode implant and for better AI data collection and processing. Despite advancements in neuroscience and technology, existing solutions remain inadequate in delivering precise, adaptive, and scalable neuro-technologies for both clinical and enhancement applications. This gap underscores the need for innovative, data-driven solutions that can harness the complexity of the brain to improve outcomes for patients and enhance the capabilities of healthy individuals.

### BRIEF SUMMARY OF THE INVENTION

In view of the foregoing issues, Applicant proposes an AI-powered neuroplasticity device via wearable and embedded brain interfaces. AI-Powered Neuroplasticity via Wearable and Embedded Brain Interfaces represents a groundbreaking intersection of neuroscience, artificial intelligence, and advanced hardware technologies. The goal is to harness the brain's natural ability to rewire itself, termed, neuroplasticity, by using adaptive AI to analyze, decode, translate, predict, and modulate neural patterns and deliver, in real-time, temporospatial, personalized, and adaptive feedback via wearable or embedded brain-computer interfaces (BCIs). This approach has the potential to revolutionize and empower an individual's brain health, cognitive augmentation, and rehabilitation.

In a first aspect, embodiments of the present disclosure relate to an AI-Powered Neuroplasticity System. The system includes a wearable or implantable Brain-Computer Interface (BCI) configured to monitor, decode, and modulate neuronal activity in real time. Further, the system includes one or more generative AI algorithms, such as Convolutional Neural Networks (CNNs), Spiking Neural Networks (SNNs), Graph Neural Networks (GNNs), and Bayesian Neural Networks (BNNs), that analyze neural signals and generate synthetic neuronal firing patterns. Further, the system includes a feedback loop for adaptive neurostimulation, dynamically adjusting electromagnetic, ultrasonic, or neurochemical stimulation based on real-time brain activity.

In a second aspect, embodiments of the present disclosure relate to a method for treating neurodegenerative diseases, such Alzheimer's, Parkinson's, and ALS. In the method, dysfunctional neuronal activity is detected through a non-invasive or implantable BCI. Predictive neuroplasticity models are generated using AI-powered neural networks, and personalized neurostimulation is delivered to promote synaptic repair and circuit reorganization.

In a third aspect, embodiments of the present disclosure relate to a system for human cognitive augmentation. The system includes a wearable AI-driven neurostimulation device designed to enhance learning, memory, and skill acquisition. The system also includes at least one adaptive signal processing algorithm that detect and reinforce beneficial neural activity patterns. Further, the system includes one or more real-time feedback mechanisms to improve cognitive and motor performance.

In a fourth aspect, embodiments of the present disclosure relate to a non-invasive neurostimulation wearable in which the wearable integrates bioelectronic sensors that detect brainwave activity and autonomously adjust stimulation parameters in which AI dynamically modulates stimulation based on real-time neural feedback.

In a fifth aspect, embodiments of the present disclosure relate to a magnetic-driven nanobot system for targeted neurostimulation in which the nanobots navigate the brain using external magnetic fields, in which the system precisely places functionalized electrodes at specific brain regions, and in which the biocompatible nanobot structure minimizes immune response and tissue damage.

In a sixth aspect, embodiments of the present disclosure relate to a Brain Interface Electrode (BIE) system. The BIE system includes flexible, conformable, and biocompatible electrodes composed of at least one of silk, graphene, or gold nanoparticles. The BIE system also has magnetically guidable properties for precise positioning within the brain and a high-fidelity signal transmission for optimized neurofeedback.

In a seventh aspect, embodiments of the present disclosure relate to an AI-powered neural activity analysis system in which AI models interpret real-time EEG, fMRI, or neural spike train data and in which the system predicts and adjusts neurostimulation patterns for personalized therapy.

In an eighth aspect, embodiments of the present disclosure relate to a neuroplasticity-driven rehabilitation method in which AI-powered BCIs detect and stimulate specific brain regions for motor and cognitive recovery after trauma, stroke, or spinal cord injury and in which the system continuously adapts based on individual neural responses.

In a ninth aspect, embodiments of the present disclosure relate to a scalable neurotechnology platform in which the AI-powered BCIs integrate with wearable or implanted devices for broad clinical and consumer applications and in which the system offers cloud-based analytics for longitudinal neural data tracking and optimization.

In a tenth aspect, embodiments of the present disclosure relate to a biocompatible, non-antigenic electrode implant for long-term neurostimulation in which the implant reduces inflammation and scarring compared to conventional electrode implants and in which the electrode adapts to brain tissue microenvironments for prolonged stability and function.

In an eleventh aspect, embodiments of the present disclosure relate to a biocompatible nanobot system for neural stimulation and repair in which the nanobots are composed of biocompatible and non-toxic materials that minimize immune response and inflammation and in which the nanobots integrate functionalized surfaces that promote neuron adhesion and synaptic connectivity, enabling targeted neural regeneration.

In a twelfth aspect, embodiments of the present disclosure relate to a dual-mode nanobot navigation system for precise neural targeting in which the nanobots are guided using a combination of external magnetic fields and cerebrospinal fluid flow, in which the system leverages fluid dynamics within the brain's ventricles and subarachnoid spaces to transport nanobots to specific neural regions, and in which the coordinated control of magnetic fields and natural fluid movement allows for high-precision delivery with minimal energy input.

In a thirteenth aspect, embodiments of the present disclosure relate to a flexible, conformable, guidable BIE (brain interface electrode), including a biocompatible material such as naturally occurring and non-antigenic protein (e.g., silk) or inert nanoparticles (e.g., gold coating, graphene). The BIE is composed of (1) a carrier (e.g. super protein/trolly/silk/biocompatible) and (2) wire for unimpeded information flow. The wire is connected to an external transducer. The carrier serves as a vehicle to direct the wire to the intended neuronal targets, and the carrier is guided by a range of different signals, including tunable magnetic fields.

In one or more embodiments, the carrier can be made of any biocompatible, biopolymeric, or inert anisotropic material with low immunogenicity. Examples of such materials include polysaccharides, lipids, synthetic hybrid biopolymers, hydrophilic polyethylene glycol (PEG) and hydrophobic poly(lactide-co-glycolide) (PLGA), hydrogel such as PEGDA/GelMA hydrogel, or other biocompatible material and contain proteins, including silk fibroin, keratin, collagen, defined secondary structural proteins (e.g. alpha-helix proteins) that are inherently magnetizable or “doped” to be magnetizable. Biocompatible magnetic nanoparticles (BMN) include super paramagnetic iron oxide, for example. The carrier facilitates the interface between specific cells (i.e. neurons) and the wire by leveraging physiochemical molecular forces.

In one or more embodiments, the carrier may use monoclonal antibodies to target specific proteins or other molecules that are natively expressed by neurons, other CNS cell types, or extracellular complexes (e.g. plaques comprised of different amyloid species).

In one or more embodiments, the carrier may have growth factors, peptides, transmitters, cytokines, and/or hormones (or any synthetic or natural ligand) attached thereto that bind to receptors or other proteins. Alternatively or additionally, the carrier may include a chemical that has tropism, attraction or affinity to structural or membrane-expressed components of CNS cells. Alternatively or additionally, the carrier may include a molecule that has attraction to molecules released by these cells, operating by chemotactic or molecular gradient. In one or more embodiments, the carrier may have a fluorescent/bioluminescent producer and photo-attractive molecule. The carrier/molecular complex may be attracted or guided to the target based on any of these forces, and the targeted cell/extracellular complex may natively express the target of these forces or induced to express them by transgenic manipulation.

In one or more embodiments, the wire provides continuous electrical connectivity to the external transducer. In such embodiments, the wire is composed of a highly flexible biocompatible material, for instance, silk fibroin, keratin, collagen or other materials that have electrical conductivity or can be doped to produce electrical conductivity. Further, in embodiments, the wire can also be composed of magnetizable frames that transmit mechanical forces that can be transduced to neurochemical or biochemical signals.

In a fourteenth aspect, embodiments of the present disclosure relate to a BCI support and interface commercial available wearable technology such as smart watches, smart headsets, smart wristbands, smart patches and more.

Other aspects, objectives and advantages of the invention will become more apparent from the following detailed description when taken in conjunction with the accompanying drawings.

### DETAILED DESCRIPTION OF THE INVENTION

Various embodiments of the present disclosure relate to a new class of neural interface systems configured to. As will be described more fully below, embodiments of the presently disclosed system provide a nanobot-based neural interface in which flexible, biocompatible electrodes are delivered via nanobots, minimizing immune response and providing long-term signal fidelity. Further, embodiments of the presently disclosed system utilize Physics-AI engine (AxonBolts), a proprietary software available from the assignee of the present application, that models neural activity as a dynamical system, learns causal relationships, and computes target neural states. Embodiments of the system implement closed-loop neural control, including real-time sensing followed by modeling and then stimulation, and adaptive neuroplasticity induction. Applicant envisions that the disclosed system may be used for drug discovery as well as stimulation, such as by simulating neural and drug interaction, predicting outcomes, and generating synthetic datasets.

These and other aspects and advantages will be discussed more fully below in relation to the embodiments provided herein. These embodiments are presented by way of illustration and not limitation.

### Wearable Brain-Computer Interfaces (BCIs):

Embodiments of the present disclosure relate to a generative AI (gen-AI) platform that integrates Convolutional Neural Networks (CNNs) with Spiking Neural Networks (SNNs) to create synthetic neuronal firing data. In one or more embodiments, the gen-AI platform employs advanced computational models to simulate the electrical activity of neurons by generating spike trains that align with well-established neuronal firing patterns. By replicating these patterns, the system can produce vast amounts of high-quality synthetic neuronal data. This abundance of data is critical for training advanced models, including Graph Neural Networks (GNNs) and Bayesian Neural Networks (BNNs). These models analyze and predict specific neuronal firing patterns, enabling precise computation of desired neural activity to induce localized neuroplasticity.

In one or more embodiments, the induced localized neuroplasticity can address two critical objectives:
1. Treatment of Neurodegenerative Diseases: Tailored neural activity can help restore or compensate for the loss of cognitive or motor neuron function by stimulating the formation of new synaptic connections and rewiring neural circuits. Applicant envisions that this approach has the potential to treat conditions like Alzheimer's, Parkinson's, and ALS, slowing disease progression and improving a patient's quality of life.
2. Human Augmentation: By inducing specific neuroplasticity, Applicant envisions that the system can enhance cognitive learning or physical capabilities. This application opens possibilities for augmenting memory, accelerating skill acquisition, and boosting physical performance, providing benefits in education, professional training, sleep, and sports performance.

To precisely apply these advancements, embodiments of the present disclosure relate to a Brain-Computer Interface (BCI) system leveraging cutting-edge conformable, functionalized electrodes. These electrodes are designed to integrate seamlessly with brain tissue, minimizing invasiveness and maximizing long-term compatibility. In one or more embodiments, placement and operation of these electrodes are facilitated by a magnetic-driven nanobot system. In such embodiments, the nanobots are designed to navigate the complex architecture of the brain, targeting specific regions with remarkable precision.

In one or more embodiments, non-invasive biosensors provide for real time data collection and monitoring for precision diagnostic feedback in neuronal networks and ultrastructural regions in the brain as outlined below. In one or more embodiments, Applicant's neuroAI toolkit can be comprised of/will have: (1) Biomolecules (proteins, such as polymers and/or silk, and antibodies, such as monoclonal and/or fragments), (2) Biocompatible Magnetic Nanoparticles and (3) Magnetizable Frames that can control both the biosensor size and magnetic or other biophysical properties along the electromagnetic spectrum. Applicant's AIM (AI Milieu) contains virtual models of physical systems to simulate and optimize the behavior of magnetic guided proteins in various neural environments. With the variables of field strength and direction, precise and localized control will minimize or eliminate effects on surrounding tissue.

The nanobots deliver the functionalized electrodes to predetermined brain regions, ensuring their accurate placement to stimulate targeted neuroplasticity. Furthermore, these nanobots are equipped to deliver precisely controlled electromagnetic activity to the target area. In one or more embodiments, this controlled stimulation is configured to provide (1) localized neuroplasticity and (2) safe and efficient stimulation. With respect to the first point, specific brain areas can be targeted with precise electromagnetic pulses to facilitate the formation or modification of neural pathways, tailored to individual needs. With respect the second point, the magnetic-driven nanobots are expected to reduce risks associated with traditional invasive procedures while improving the accuracy and effectiveness of treatment.

By combining the capabilities of gen-AI, advanced modeling techniques, and cutting-edge BCIs, embodiments of the presently disclosed integrated solution are expected to provide a transformative approach to treating neurodegenerative diseases and augmenting human capabilities. It leverages synthetic data generation, machine learning-driven insights, and precision-engineered nanotechnology to unlock unprecedented possibilities in neuroscience and neurotechnology.

### Non-Invasive and Wearable Neurostimulation Devices

In one or more embodiments, the presently disclosed system introduces non-invasive, wearable devices capable of detecting and influencing brainwave activity seamlessly. These devices can take the form of neurostimulation headbands, brain-sensing earphones, neurostimulation pillows, or wearable garments (e.g., T-shirts). Advantageously, the wearable devices are designed to blend seamlessly into daily life, ensuring continuous monitoring of brainwave activity without disrupting routine activities. The devices integrate advanced sensors for real-time detection of neural activity and transmit the data wirelessly and bidirectionally to the associated AI system.

### AI-Powered Neural Activity Analysis and Feedback Mechanism

In one or more embodiments, robust AI algorithms (including CNNs, SNNs, GNNs, and BNNs) analyze neural activity, offering real-time, highly granular insights into how the brain is functioning and adapting. The algorithms are capable of identifying specific patterns of dysfunction or underactivity in brain regions. This analysis drives precision neurofeedback, which targets specific brain areas to encourage neuroplastic changes, such as improving synaptic connections, repairing damaged circuits, or enhancing cognitive and motor performance. Advantageously, this AI-driven feedback loop allows for dynamic, adaptive stimulation tailored to the user's specific neural needs, enhancing the efficacy of neuroplasticity interventions.

In one or more embodiments, the AI-powered neural activity is controlled by Physics-AI engine (“AxonBolts”), a proprietary software available from the assignee of the present application, which models neural activity as a time-evolving dynamical system. AxonBolts is configured to learn neural behavior, predict outcomes, and generate intended neural activity states. Such functions may be beneficial for simulating drug interactions, predicting neural responses, and accelerating validation of therapies.

### Flexible, Conformable, and Guidable Brain Interface Electrodes (BIE)

In one or more embodiments, the presently disclosed system incorporates Brain Interface Electrodes (BIE) constructed from biocompatible materials such as naturally occurring, nonantigenic proteins (e.g., silk) or inert nanoparticles (e.g., gold coatings, graphene). Advantageously, such electrodes are flexible and conformable, designed to integrate seamlessly with brain tissue without causing damage or inflammation. Further, the electrodes may be magnetically guidable by functionalizing the electrodes with tunable magnetic fields to enable precise navigation and placement within the brain, even in complex or deep regions. In embodiments, the electrodes provide high-fidelity data transmission through the inclusion of conductive wiring or graphene layers for unimpeded and efficient information flow between external transducers and targeted brain areas. Applicant expects that this innovative design will minimize invasiveness while ensuring accuracy and long-term compatibility.

In accordance with the foregoing discussion, the BIE can be formed from a diverse array of biopolymers, biomolecules, and naturally occurring proteins exhibit magnetizable or electromagnetic orientation properties. These include iron-storage and magnetotactic proteins, nucleic acids, cytoskeletal filaments, structural biopolymers, and engineered protein-based nanomaterials. Their mechanisms range from intrinsic magnetism and dielectrophoresis to diamagnetic alignment and piezoelectricity. Examples of magnetizable (Fe storage or magnetotactic) proteins include ferritin, hemoglobin, myoglobin, and magnetosome-associated proteins that are naturally magnetic or magnetizable, enabling orientation and manipulation in magnetic fields. Electro-orientable biomolecules (“filamentous) include nucleic acids (DNA/RNA), actin, collagen, fibrin, oriented by electric fields via dielectrophoresis or electroorientation. Diamagnetic alignment (structural proteins) include silk fibroin, fibrin, collagen, tubulin, and cellulose nanocrystals that align in stronger magnetic fields due to diamagnetic anisotropy. Piezoelectric biopolymers include silk, collagen, keratin, cellulose, PHAs, and PLA that can be oriented by electric fields. Cytoskeletal/motor proteins include tubulin, actin, and motor proteins that can be oriented or manipulated with intrinsic electromagnetic properties and field-induced orientation. Engineered protein-based nanomaterials may have intrinsic electromagnetic properties and field-induced orientation.

Integration with Wearable Technology for Continuous Monitoring and Stimulation

The above-described BIE technology is seamlessly combined with wearable devices to provide a holistic, real-time neurotechnology platform. Wearables equipped with embedded sensors communicate with the BIE system, creating a feedback loop for continuous monitoring and neural stimulation. Advantageously, this integration allows users to benefit from neural optimization during their daily routines, providing long-term, tailored neuroplasticity stimulation without requiring frequent medical interventions.

### Magnetic-Driven Nanobot System for Targeted Delivery

In one or more embodiments, a magnetic-driven nanobot delivery system ensures the precise placement of functionalized electrodes at targeted brain regions. The nanobot system leverages biocompatible proteins, such as (but not limited to) silk, that may be functionalized to be magnetically responsive, e.g., by incorporating ions (such as ferric ions or other magnetic elements). The ability to use customizable proteins ensures adaptability for different biomedical applications while maintaining safety and minimizing risks of tissue damage or inflammation. These nanobots are expected to navigate the brain's complex structure with unparalleled precision, delivering conformable electrodes or specific electromagnetic pulses to induce desired neuroplasticity. As mentioned, this approach is expected to reduce the risks associated with traditional invasive techniques, offering a minimally invasive, efficient, and safe alternative.

### Applications for Neurodegenerative Diseases and Human Augmentation

Embodiments of the presently disclosed system have multiple applications, including for neurodegenerative diseases and human augmentation. In relation to neurodegenerative diseases, the system allows for treatment of conditions such as Alzheimer's, Parkinson's, and ALS by repairing neural circuits, improving synaptic connections, and compensating for lost neural function. For human augmentation, the system is configured to enhance learning, memory, or motor performance, providing benefits for education, training, and professional or athletic performance.

Embodiments of the system incorporate energy-efficient neurostimulation methods that dynamically adjust the intensity and frequency of stimulation to minimize energy consumption while maximizing neuroplasticity outcomes. This optimization ensures the safety and longevity of both the wearable devices and the internal BIE components.

Further, by integrating data-driven, personalized, and precision neurotechnology, the system is expected to improve significantly the quality of life for users. The system is configured to ensure long-term compatibility with individual needs, allowing for ongoing cognitive, emotional, or motor enhancements without the need for invasive procedures.

As discussed above, “BIE” stands for Brain Interface Electrode. According to the present disclosure, the BIE is a cutting-edge device designed to interact with the brain for neurostimulation, monitoring, or other neural activities. In one or more embodiments, the BIE has one or more of the following features: flexible and conformable design, magnetic guidability, high-fidelity data flow, non-antigenic properties, and customizable functionalization. In this regard, the BIE is made from biocompatible materials (e.g., silk proteins, graphene, or gold nanoparticles), allowing it to integrate seamlessly with brain tissue without causing damage or inflammation. Further, the conformable nature means it can adapt to the brain's complex, soft structure.

In embodiments, the BIE is functionalized with magnetic responsiveness so that the BIE can be precisely guided to specific regions of the brain using external magnetic fields. Advantageously, this feature is expected to minimize the need for invasive surgery.

In embodiments, the electrode ensures unimpeded and efficient information transmission between external devices (e.g., AI systems or neurostimulators) and the targeted brain regions. Further, the BIE leverages materials like naturally occurring proteins (e.g., silk) or inert nanoparticles that do not trigger an immune response, ensuring long-term compatibility. Additionally, the electrode can be functionalized for specific purposes, such as delivering precise electromagnetic activity or interacting with particular neural circuits to induce neuroplasticity.

Applicant envisions several potential use Cases of the BIE. For example, the BIE may be used for neurodegenerative disease treatment by repairing or compensating for damaged neural pathways. In another example, the BIE may be used for human augmentation by enhancing cognitive or motor skills for learning, physical performance, or professional tasks. In still another example, the BIE may be used for real-time monitoring by detecting and analyzing brainwave activity for adaptive feedback and optimization.

Wearable BCIs can be used for various applications, such as restorative or enhancing neural cognition, mental health improvement, stress management, and recovery from neurodegenerative-associated with aging, trauma, and brain injuries. Embodiments of the presently disclosed embedded Brain-Computer Interfaces (BCIs) utilize Applicant's biocompatible BRAIN_{[MOU1]}N (Biocompatible Restorative Artificial Intelligence Neuroplasticity Network) approach through SMART_{[MOU2]}(Structured Magnetic AI Radiofrequency Transduction) technology. Embedded BCIs are more invasive and may involve implanting electrodes or microchips in the brain to directly interface with neural networks. These devices are typically used in more severe cases, such as for patients with neurological disorders (e.g., Parkinson's disease, epilepsy, or paralysis). AI-powered systems can interact with embedded BCIs to continuously monitor neural activity, predict changes, and provide targeted neurostimulation, helping to restore lost functions or improve brain health over time.

According to the present disclosure, AI can play a role in neuroplasticity. For example, AI systems can employ machine learning, especially Bayesian Neural Networks (BNNs) and Graph Neural Networks (GNNs), to interpret complex brain signals, identifying patterns that are linked to learning, cognitive functions, or disorders. Further, the AI systems can be used for personalized neurostimulation. In particular, by understanding neural patterns, AI can optimize the type, intensity, and timing of neurostimulation delivered through wearable or embedded BCIs, encouraging specific brain areas to reorganize and adapt. Still further, AI systems can be used for continuous learning from the user's own brain data, modulating the interventions to enhance neuroplasticity based on real-time feedback derived insights.

Applications of AI-Powered Neuroplasticity include cognitive enhancement, rehabilitation, mental health, and brain health and aging. With respect to cognitive enhancement, wearable BCIs combined with AI allow users to train their brains for enhanced focus, memory, learning ability, and creativity. These devices can be used by healthy individuals to boost cognitive performance in personal or professional settings. For rehabilitation, AI-powered BCIs offer powerful tools for neurorehabilitation following traumatic brain injuries (TBI), strokes, or spinal cord injuries. By stimulating brain regions involved in motor control or cognitive functions, they help patients regain lost skills through accelerated neuroplastic changes. Embedded BCIs can also aid individuals with severe motor impairments or neurological disorders by restoring communication between the brain and paralyzed limbs. In the field of mental health, AI-driven neurostimulation via wearables can be used to manage conditions such as depression, anxiety, and PTSD. Through the high-resolution mapping of the brain's emotional and cognitive pathways and promotion of a positive neuroplastic response, thereby improving mental well-being. Regarding brain health and aging, continuous brain monitoring through BCIs serves to surveil and detect early signs of mental health issues, providing preventative care through tailored neurostimulation. For older adults, AI-powered BCIs can monitor and predict preclinical signs of cognitive decline to improve memory, attention, and overall brain function by stimulating neural pathways associated with learning and memory.

Based on the forgoing, AI-powered neuroplasticity via non-invasive BCIs offers many advantages. One such advantage is non-invasive monitoring and stimulation. Wearable BCIs allow for safe, real-time brain analysis and neurostimulation without the need for surgery or hospitalization. Another advantage is precision, personalization, and real time adaptation. AI customizes interventions based on each individual's brain activity, offering highly effective and targeted neuroplastic stimulation for maximizing health and quality of life. Still another advantage is scalability and universality because wearable BCIs can be used by a wide range of individuals—from patients recovering from neurological damage to healthy users looking for cognitive enhancement—making the technology accessible to a broad market.

As such, Applicant envisions that the combination of AI and BCIs driving neuroplasticity in health and disease, is poised to transform how brain-related conditions are treated and to enhance cognitive capabilities. Applicant expects that wearable devices will become more mainstream, allowing for daily brain training or mental health improvement. Embedded BCIs, though still primarily used for clinical purposes, could advance into more sophisticated, less invasive forms that offer lifechanging benefits to patients with neurological impairments.

AI-powered neuroplasticity via BCIs is a field that not only promises to improve brain health but also redefines the boundaries of human potential. By enabling the brain to adapt and improve continuously, Applicant believes that the world is moving into a new era of cognitive enhancement and personalized brain care.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A system for modulating neural activity in a subject, comprising:
a neural interface comprising at least one sensor configured to detect electrophysiological signals from the subject;
a processor operatively coupled to the neural interface;
a machine learning model executed by the processor and configured to receive the electrophysiological signals and identify one or more neural activity patterns;
a stimulation module configured to deliver electrical, magnetic, or electromagnetic stimulation to the subject;
wherein the processor is configured to generate stimulation parameters based on the identified neural activity patterns and control the stimulation module in real time based on feedback from the detected electrophysiological signals.

2. The system of claim 1, wherein the neural interface comprises a wearable device positioned on the head of the subject.

3. The system of claim 1, wherein the neural interface comprises an implantable electrode.

4. The system of claim 1, wherein the machine learning model comprises at least one of a convolutional neural network, a spiking neural network, or a graph neural network trained on neural signal data.

5. The system of claim 1, wherein the electrophysiological signals comprise at least one of EEG signals, local field potentials, or neural spike data.

6. The system of claim 1, wherein the stimulation module delivers stimulation having at least one adjustable parameter selected from amplitude, frequency, and pulse width.

7. A method for modulating neural activity in a subject, comprising:
acquiring electrophysiological signals from the subject using a neural interface;
inputting the electrophysiological signals into a machine learning model executed by a processor;
identifying one or more neural activity patterns associated with a target neural state;
generating stimulation parameters based on the identified neural activity patterns;
delivering stimulation to the subject using a stimulation module; and
updating the stimulation parameters in real time based on feedback from subsequent electrophysiological signals.

8. The method of claim 7, wherein the electrophysiological signals are acquired using signals from the implanted electrodes.

9. The method of claim 7, wherein delivering stimulation promotes synaptic plasticity in a targeted brain region.

10. The method of claim 7, wherein the machine learning model is updated using reinforcement learning based on the feedback.

11. A neural interface device comprising:
a plurality of electrodes configured to detect electrophysiological signals;
a signal processing module configured to filter and extract features from the electrophysiological signals;
a communication module configured to transmit the extracted features to a processor that determines stimulation parameters.

12. The device of claim 11, wherein the electrodes comprise a flexible biocompatible material selected from at least one of biopolymers, biomolecules, and naturally occurring proteins exhibiting magnetizable or electromagnetic orientation properties.

13. A system for generating neural stimulation parameters, comprising:
a processor configured to execute a machine learning model trained on electrophysiological signal data;
wherein the machine learning model outputs stimulation parameters that are used to control a stimulation device configured to deliver stimulation to neural tissue.

14. The system of claim 1, wherein the system is configured to treat a neurological disorder or enhance cognitive performance.

15. A system for modulating neural activity comprising:
a neural interface;
a processor;
a machine learning and Physics-AI model;
a stimulation module;
a plurality of nanobots configured to deliver flexible electrode structures;
wherein the Physics-AI model represents neural activity as a dynamical system;
wherein the processor computes a target neural activity state; and
wherein stimulation is applied to induce said state.

16. The system of claim 15, wherein the electrode structures are biocompatible and minimize immune response.

17. The system of claim 15, wherein nanobots dynamically position and reconfigure electrodes.

18. The system of claim 15, wherein electrodes prevent scar tissue formation.

19. The system of claim 15, wherein the Physics-AI model learns causal relationships between stimulation and neural response.

20. The system of claim 15, wherein the system generates intended neural activity patterns.

21. The system of claim 15, wherein stimulation induces targeted neuroplasticity.

22. A system for simulating neural activity comprising:
a Physics-AI model configured to simulate neural dynamics;
wherein the model simulates interaction between neural activity and pharmacological agents and predicts neural outcomes.

23. The system of claim 22, wherein synthetic neural data is generated.

24. A method comprising:
modeling neural activity;
simulating drug interaction; and
predicting neural response.

25. The system supports wearable and implantable interfaces.

26. The system enables cognitive enhancement.

27. The system enables treatment of neurological disorders.
